(19) United States
(12) Patent Application Publication
(10) Pub. No.: US 2009/0246246 A1
(43) Pub. Date: Oct. 1, 2009

(54) PROCESSES FOR RELIEVING BLOCKED VESSELS AND PRODUCTS THEREBY

(76) Inventor: William George, Gresham, OR (US)

Correspondence Address: Luce, Forward, Hamilton & Scripps LLP, 2050 Main Street, Suite 600, Irvine, CA, 92614, US

(21) Appl. No.: 12/395,573

(22) Filed: Feb. 27, 2009

Related U.S. Application Data
(60) Provisional application No. 61/032,740, filed on Feb. 29, 2008.

(51) Int. Cl. A61F 2/00; C40B 30/00; C40B 50/00; G06F 17/30

(52) U.S. Cl. 424/423; 506/7; 506/23; 707/104.1; 707/E17.044

(57) ABSTRACT

Select microbials, microbiologicals, biologics and related naturally occurring or engineered microbial solutions supplement existing device modalities. Likewise, librarying and mapping processes are disclosed by which novel enhanced processes for relieving blocked vessels can be discovered, generated or improved for intravascular usages.

### CROSS-REFERENCE TO RELATED APPLICATIONS

This application claims the priority benefit of the U.S. Provisional Patent Application Ser. No. 61/032,740 filed Feb. 29, 2009, by the present inventor and full Paris Convention priority rights are claimed, the same being expressly incorporated by reference as if fully set forth herein.

### BACKGROUND OF THE INVENTION

1. Field

This disclosure relates to systems for addressing and relieving the deleterious impacts of blocked vessels in mammals. Particularly, this disclosure relates to systems to mitigate the harmful impact of fatty tissue/cholesterol-based plaques which occlude vessels, fully or partially, requiring further interventions and or causing death in mammals.

2. General Background

Among the most biologically diverse collection of organisms are what may loosely be termed “microbiologicals” or bacteria, viruses and other sub-microscopic flora. While these seemingly industrious and ubiquitous collections of species have been applied to tasks as varied as cleaning up oil spills, using them within the microbiological plaque-addressing world to help people overcome the challenges of blocked vessels remains a longstanding need.

Owing to the suitability of many microorganisms for just such tasks, the present inventor challenges conventional logic with his approach to enhancing the progress in science and the useful arts by using a novel and inventive strategy.

Expressly incorporated herein by reference, U.S. Pat. Nos. 6,663,667-6,849,088; 6,808,529; 7,311,726; U.S. Publication Numbers: 2003/0120263; 2005/0181059; and 2006/0229701.

The contents of the above-identified patents, publications, and all other documents mention or cited herein, are hereby incorporated by reference in their entirety. Applicants reserve the right to physically incorporate into this application any and all materials and information from any such patent applications, or other documents mentioned or cited herein.

### SUMMARY OF THE INVENTION

Briefly stated, systems are disclosed for devices and microorganism combinations which improve on the art.

According to embodiments, specially designed microbials and biologics work in combination with known stents, graphs, temporary and permanent implants and related delivery vehicles to mitigate harmful impacts of fatty tissue.

According to further embodiments, microbiological testing facilities are established to develop a database of known organisms, as discussed below, and to process then for medical use, by studying their efficacy against various disease states, hereditary conditions and medical challenges.

### DETAILED DESCRIPTION OF THE INVENTION

The present inventor has discovered that microbiological agents have been under utilized and combinations with cardiology and radiology-based devices are not present in the market place, for the cardiovascular applications for which they are needed, let alone in the peripheral vasculature and brain. The present invention addresses and ameliorates this longstanding need, and in so doing constitutes progress in science and the useful arts.

The teaching of the present invention are focused upon making vessels patent through emplaced systems of biologies, microbials and related processes for treatment of disease-state and/or age-challenged mammals.

According to embodiments, there are disclosed at least two distinct approaches to using the processes and products of the present invention, as discussed herein and claimed below. Namely, those skilled in the art understand and appreciate known and later developed compounds and biologies having efficacy can be used to replace systems currently in place, according to the instant teachings.

Other and further embodiments include disease states and target patients whose particular challenges do not make them ideal candidates for other treatments. For example, issues with existing minimally invasive systems renders the same inappropriate for many patients.

In contradistinction, the instant teachings often can be used to address situations where reperfusion and lysis are needed to be established rapidly, and long-term indwelling of medical devices (currently known) cannot be tolerated.

Likewise, within the context of database development of efficacious biologies and microbials, those skilled in the art readily understand that numerous existing and discoverable agents can be used to relieve blockages within lumens in mammals. Within the scope of the present invention are processes to harvest and library compounds which work.

The present invention contemplates and claims generating and having peer-reviewed new libraries of biologies, microbials and related compounds. By so doing, centers of excellence are created which allow those who have discovered in this field to collaborate with the instant teachings and have the same made available, further tested and accessed by clinicians and thought leaders.

The following description includes information that may be useful in understanding the present invention. It is not an admission that any of the information provided herein is prior art to the presently claimed inventions, or relevant, nor that any of the publications specifically or implicitly referenced are prior art.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

The improved processes of the present invention are effective to use with any number of conventional, or after-developed technologies. Those skilled in the art understand that treatments are needed both before, after and during placement of known tools used to create patency in vessels. Microbials, and biologics now help with these issues, according to the teachings of the present invention.

For example, during pre-placement angioplasty use of microbial agents can render a vessel wall better able to receive any conventional implant. Likewise, once in situ, can be prevented. Further, to explant or replace the '726 stent is made easier by the teachings of the instant disclosure.

Referring now to U.S. Pat. Nos. 6,849,088; 6,808,529 and 6,663,667 there are shown other aspects and features of the present invention, namely systems which work with the present teachings. Whether Aorto Uni-Iliac grafts, bare metal or drug electing stents or texturized surfaces are involved, the instant teachings function to utilize enhanced microbial activity to keep vessels patent.

Turning likewise to Publications number 2003/0120263; and 2006/0229701 further context and useful embodiments of the instant teachings are shown, by way of systems improved by the teaching of the present invention.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. In a system for generating databases of biologics and microbiological agents used for addressing disease states in mammals, the improvement comprising:
a research map for using specific microorganisms in combination with existing devices; and
a database of known organisms fulfilling basic requirements for acting on the vasculature of an animal by enhancing patency of select aspects of the vasculature thereof.

2. The system of claim 1, further comprising disease state targets.

3. The system of claim 2, further comprising at least one device from the group consisting of a stent; a stented-graft; an expandable prosthesis; and related devices used to restore, maintain or increase flow in a vessel.

4. A method for enhancing patency in vasculature, which comprises:
providing a principal device system for making a lumen less obstructed than it is;
supplementing the principal device system with at least one substance selected from the group consisting of biologics, microbiological agents, and related organisms which improve patency values.

5. The method of claim 4, wherein the supplementary step includes delivery of the at least one substance to a mammal with obstruction challenged vessels.

6. The method of claim 5, further comprising the system of claims 1.

7. The method of claim t, further comprising disease state targets.

8. A product, by the method of claim 5, offered to patients for treatment.

9. A process for creating a library of useful agents to address insults to patency in vessels, comprising, in combination:
generating a list of target substances to assess focused utility of;
testing efficacy with respect to at least a plurality of vessel blocking modalities and procuring delivery of the same with respect to a region to be treated.

10. The process of claim 9, further comprising screening at least one of a biologic and a microbial agent for efficacy relative to at least a blockage within a vessel.

11. A product by the process of claim 10.

12. A process for addressing vessel blockages in mammals, comprising, in combination:
selecting at least one of a biologic and a microbial agent; and
emplacing the same within a zone to be treated.

13. The process of claim 12, further comprising use in combination with other treatment modalities.
